(19) United States
(12) Patent Application Publication
(10) Pub. No.: US 2012/0165646 A1
(43) Pub. Date: Jun. 28, 2012

(54) COMPOSITE PARTICLE, CONTRAST AGENT FOR PHOTOACOUSTIC IMAGING, AND PHOTOACOUSTIC IMAGING METHOD

(75) Inventors: Fumio Yamauchi, Yokohama-shi (JP); Satoshi Ogawa, Kawasaki-shi (JP); Kengo Kanazaki, Yokohama-shi (JP); Tatsuki Fukui, Yokohama-shi (JP); Sachiko Inoue, Kawasaki-shi (JP)

(73) Assignee: CANON KABUSHIKI KAISHA, Tokyo (JP)

(21) Appl. No.: 13/328,802

(22) Filed: Dec. 16, 2011

(30) Foreign Application Priority Data
Dec. 27, 2010 (JP) 2010-290328

(51) Int. Cl. A61B 5/05; A61K 49/00; G01N 33/53; C07K 16/00

(52) U.S. Cl. 600/407; 530/391.3; 424/9.1; 435/7.1

(57) ABSTRACT

Regarding a composite particle in the related art, an antibody and a dye are bound to the surface of the particle. Therefore, it is not possible to bind both the antibody and the dye high in number. Accordingly, the present invention provides a composite particle, wherein a large acoustic wave can be emitted and both the antibody and the dye can be bound high in number. A composite particle including a core particle, a dye bound to the above described core particle, and an antibody, wherein the above described antibody is bound to the above described dye, is suitable for use.

### BACKGROUND OF THE INVENTION

1. Field of the Invention

The present invention relates to a composite particle, a contrast agent for photoacoustic imaging, and a photoacoustic imaging method.

2. Description of the Related Art

It is known that Resovist (registered trademark) (a plurality of iron oxide particles coated with dextran which is a polysaccharide) serving as a contrast agent for MRI emits an acoustic wave (Biomed Tech 2009; 54: 83-88, hereafter referred to as Non patent literature 1).

Meanwhile, a particle prepared by binding a fluorochrome, Cy 5.5, and an antibody to a surface of CLIO (a plurality of iron oxide particles coated with cross-linked dextran) has been known. This particle can be used for MRI and fluorescence imaging of an antigen (Bioconjugate Chem. 2005, 16, 576-581, hereafter referred to as Non patent literature 2).

However, Resovist (registered trademark) includes only iron oxide particles as an acoustic wave emission source and, therefore, an acoustic wave emitted from Resovist (registered trademark) is small. Consequently, a contrast agent for photoacoustic imaging, which emits a larger acoustic wave, has been desired. Furthermore, it is believed that Resovist (registered trademark) does not include an antibody and, therefore, is difficult to detect an antigen specifically.

Meanwhile, the particle described in Non Patent Literature 2 has a problem in that the antibody and the fluorochrome are bound to the surface of the particle and, therefore, both the antibody and the fluorochrome high in number cannot be bound.

### SUMMARY OF THE INVENTION

A composite particle according to a first aspect of the present invention includes a particle containing a first dye having a molar absorption coefficient of 10 M⁻¹cm⁻¹ or more at least one wavelength selected within the range of 600 nm to 1,300 nm, a second dye which is bound to the surface of the above described particle and which has a molar absorption coefficient of 10 M⁻¹cm⁻¹ or more at least one wavelength selected within the range of 600 nm to 1,300 nm, and an antibody, wherein the above described antibody is bound to the above described second dye.

A composite particle according to a second aspect of the present invention includes a particle containing an inorganic material, an organic dye bound to the above described particle, and an antibody, wherein the above described antibody is bound to the above described organic dye.

The composite particle according to aspects of the present invention includes the second dye in addition to the first dye as the agent to emit an acoustic wave. Therefore, a large acoustic wave can be emitted. Furthermore, the composite particle according to aspects of the present invention includes both the antibody and the second dye high in number because the antibody can be bound to the second dye.

Further features of the present invention will become apparent from the following description of exemplary embodiments with reference to the attached drawings.

### BRIEF DESCRIPTION OF THE DRAWINGS

FIG. 1 is a schematic diagram for explaining a composite particle according to an embodiment of the present invention.

FIG. 2 is a schematic diagram showing a method for preparing a composite particle according to an embodiment of the present invention.

FIG. 3A is a graph showing the relationship between the number of moles of fed Dye A and the number of moles of bound Dye A in an example according to the present invention, and FIG. 3B is a graph showing the relationship between the number of moles of bound Dye A and the fluorescence intensity.

FIG. 4A is a graph showing the relationship between the number of moles of fed Dye B and the number of moles of bound Dye B in an example according to the present invention, and FIG. 4B is a graph showing the relationship between the number of moles of bound dye B and the photoacoustic signal intensity.

FIGS. 5A and 5B are graphs showing the photoacoustic signal intensities of NP-2-395-IgG and the like in examples according to the present invention.

### DESCRIPTION OF THE EMBODIMENTS

The embodiments according to the present invention will be described below. However, the present invention is not limited to these embodiments.

### First Embodiment

A composite particle according to the present embodiment includes a particle (hereafter may be referred to as a core particle) containing a first dye having a molar absorption coefficient of 10 M⁻¹cm⁻¹ or more at least one wavelength selected within the range of 600 nm to 1,300 nm, a second dye which is bound to the surface of the above described particle and which has a molar absorption coefficient of 10 M⁻¹cm⁻¹ or more at least one wavelength selected within the range of 600 nm to 1,300 nm, and an antibody, wherein the above described antibody is bound to the above described second dye.

The composite particle according to the present embodiment includes the second dye in addition to the first dye. Therefore, light is absorbed to a great extent, and a large acoustic wave can be emitted. Meanwhile, the antibody can be bound to a part of or all the second dye bound to the particle 102. In the case where the second dye has a plurality of binding site, to which the antibody can be bound, a plurality of antibodies can be bound to one second dye. The composite particle includes both the antibody and the second dye high in number by employing such a form. As a result, when the composite particle is irradiated with light, a large acoustic wave can be emitted and an antigen region can be detected more accurately.

In the case where the second dye is not bound to all binding sites, to which the second dye can be bound, on the surface of the particle, the antibody may be bound to the binding sites, to which the second dye is not bound. Furthermore, another antibody may be bound to the antibody. The antibody and another antibody may be the same or be different.

The composite particle, in which the second dye is bound to the particle containing the first dye and the antibody (not emitting an acoustic wave) is bound to the second dye, emits an acoustic wave larger than that of the composite particle, in which the second dye is bound to the particle containing the first dye. The reason for this is believed to be that in the case where the antibody is bound to the second dye, the excitation energy obtained by irradiating the second dye with the light is not emitted as fluorescence easily. That is, when the antibody is bound to the second dye, at least a part of the excitation energy of the second dye is converted to vibration and rotation energy of the antibody or thermal energy of a surrounding medium. It is believed that at least a part of the vibration and rotation energy of the antibody is transferred to the first dye and the surrounding medium and is consumed as thermal energy, so as to be emitted as an acoustic wave. As a result, it is believed that in the case where the antibody is bound to the second dye, a large acoustic wave is generated as compared with the case where the antibody is not bound.

In the present embodiment, the first dye and the second dye are not specifically limited insofar as the molar absorption coefficients are 10 M⁻¹cm⁻¹ or more at least one wavelength selected within the range of 600 nm to 1,300 nm. The molar absorption coefficient may be 10⁵ M⁻¹ cm⁻¹ or more at least one wavelength selected within the range of 600 nm to 1,300 nm, such as 10⁷ M⁻¹cm⁻¹ or more, and even 10⁸ M⁻¹cm⁻¹ or more. In the case where the molar absorption coefficient is large, the intensity of the emitted acoustic wave increases, and a higher signal intensity can be obtained in a photoacoustic imaging method. This is because the molar absorption coefficient and the intensity of the acoustic wave are in a proportional relationship.

As for the first dye and the second dye in the present embodiment, inorganic materials and organic dyes may be used.

As for the core particle in the present embodiment, a particle including at least one of the inorganic material and the organic dye may be used. However, a particle containing the inorganic material can be used.

The first dye and the second dye in the present embodiment can be different from each other. Furthermore, the first dye can be the inorganic material and the second dye can be the organic dye.

Inorganic Material

Examples of inorganic materials in the present embodiment may include metal oxides, metals, and other inorganic materials.

Examples of the above described metal oxides may include iron oxide (Fe₂O₃, Fe₃O₄), magnesium oxide, aluminum oxide, silicon dioxide, zinc oxide, titanium oxide, zirconium oxide, manganese oxide, and boron oxide. Examples of the above described metals may include gold, silver, copper, and platinum. Furthermore, a colloid by hybridization of gold, silver, copper, and platinum may be used. Examples of other inorganic materials may include cadmium sulfide, zinc selenide, cadmium selenide, zinc telluride, cadmium telluride, zinc sulfide, lead sulfide, carbon black, fullerene, carbon nanotube, and iron oxalate. As for the inorganic material, a particle of iron oxide (hereafter may be referred to as iron oxide particle) having absorption in a wavelength region of 600 nm to 1,300 nm can be used.

The shape of the inorganic material according to the present embodiment is not specifically limited, and examples thereof include the shapes of a sphere, a nanorod, a nanocube, a nanoprism, and a nanoshell. The inorganic material may be present in the state of a dry particle or be present in the state of colloid in a liquid.

Particle Diameter of Inorganic Material

In the present invention, the particle diameter of the inorganic material is determined by measuring the diameter of an image of the inorganic material taken with a transmission electron microscope (TEM). In the case where the image of the inorganic material is not in the shape of a sphere, the lengths of the short axis and the long axis of the image of the inorganic material are measured and an average value of them is taken as a diameter.

In the present embodiment, the particle diameter of the inorganic material may be 15 nm or more and 500 nm or less, and even 20 nm or more and 500 nm or less.

Iron Oxide Particle

The iron oxide particle in the present embodiment is not specifically limited, and for example, a particle formed from Fe₃O₄ (magnetite), γ-Fe₂O₃ (maghemite), or a mixture thereof may be used. It is known that the magnetite has a molar absorption coefficient higher than the molar absorption coefficient of the maghemite in a wavelength region of 600 nm to 1,300 nm, and it is believed that an emitted acoustic wave is larger than the acoustic wave of the maghemite. Therefore, the iron oxide can be the magnetite.

Meanwhile, the iron oxide particle in the present embodiment may be in the crystal state of any one of single crystal, polycrystal, and amorphous.

As for the iron oxide particle in the present embodiment, a commercially available iron oxide particle may be used, or the iron oxide particle obtained by the following method may be used. Examples of the methods include a method in which a solution is prepared by dissolving FeCl₃ and FeCl₂ into water and ammonia water is added while the resulting solution is agitated, so as to obtain the iron oxide particle.

Many particles including the iron oxide particle are commercially available, and a person skilled in the art can get and use an appropriate particle easily. Meanwhile, there are many literatures related to a method for manufacturing the particle including the iron oxide particle, and the particle can be synthesized easily in consultation with them. For example, an aqueous solution prepared by mixing and dissolving FeCl₃.6H₂O (25.5 g) and FeCl₂ 4H₂O (10.2 g), powdered dextran (molecular weight 10,000 dalton, 360 g), and 30% NH₄OH solution (30 ml) are used, and a colloidal solution of dextran particles including iron oxide particles is prepared on the basis of the method of U.S. Pat. No. 5,262,176. Furthermore, it is also possible to bind various linker molecules to a particle including the iron oxide particle. For example, 1-ethyl-3-(3-dimethylaminopropyl)carbodiimide hydrochloride (3.6 mg) serving as a carbodiimide condensation agent and 3,6-dioxaoctanedioic acid (3.6 mg) are dissolved with 0.5 M β-morpholinoethane sulfonic acid buffer solution (1.25 ml, pH=6.3). The resulting solution are incubated at 50° C. for 10 minutes and is added to the above described dextran particle colloidal solution. A reaction is effected at room temperature for 2 hours and dextran particles are refined with a magnet, so as to obtain dextran particles in which oligoethylene oxide serves as a linker molecule and a carboxyl group is included at an end thereof.

The iron oxide particle included in the composite particle according to the present embodiment may be a primary particle or a secondary particle. Regarding the iron oxide particle in the present embodiment, the surface of the primary particle or the surface of the secondary particle may be covered with a surface-modifying agent.

Particle Diameter of Iron Oxide Particle

In the present invention, the particle diameter of the iron oxide particle is determined by measuring the diameter of an image of the iron oxide particle taken with a transmission electron microscope (TEM). In the case where the image of the particle is not in the shape of a sphere, the lengths of the short axis and the long axis of the image of the iron oxide particle are measured and an average value of them is taken as a diameter.

In the present embodiment, the particle diameter of the iron oxide particle may be 15 nm or more and 500 nm or less, and even 20 nm or more and 500 nm or less.

The composite particle according to the present embodiment may include only one iron oxide particle or include at least two iron oxide particles. In the case where the particle diameter of the iron oxide particle included in the composite particle is 500 nm, the number of iron oxide particles included in the composite particle may be 3 or less.

Particle Containing Inorganic Material

In the present embodiment, the particle containing the inorganic material is not specifically limited insofar as the particle absorbs light and emits an acoustic wave. Here, the light refers to ultraviolet light (an electromagnetic wave with a wavelength of 10 nm to 400 nm), visible light (an electromagnetic wave with a wavelength of 400 nm to 600 nm), near-infrared light (an electromagnetic wave with a wavelength of 600 nm to 1,300 nm), and the like. In the present embodiment, the particle containing the inorganic material can be the particle which absorbs near-infrared light and emits an acoustic wave.

The particle containing the inorganic material, according to the present embodiment, refers to any one of (1) a particle formed from only the inorganic material, (2) a particle formed by dispersing the inorganic material in an in organic substance or an organic substance, and (3) a particle formed by coating the inorganic material with an in organic substance or an organic substance. In the present embodiment, any one of the items (1) to (3) or a combination thereof may be used.

In the present embodiment, it is essential that the particle containing the inorganic material contains at least one inorganic material described later, and at least two types may be contained. In this regard, it is essential that the number of inorganic material in the particle containing the inorganic material is at least one, and the number may be at least two.

As an example of the particle containing the inorganic material, a particle formed from iron oxide covered with dextran is considered. At this time, the weight ratio of dextran to iron oxide (dextran (g)/iron oxide (g)) may be within the range of 0.1 to 10, and even within the range of 1 to 5. It is believed that in the case where the weight ratio of dextran to the iron oxide is 0.1 or more, the iron oxide is not exposed at the surface of the particle easily and the dispersion stability of the particle is good.

Shape of Particle Containing Inorganic Material

In the present embodiment, the shape of the particle containing the inorganic material is not specifically limited. Examples thereof include the shapes of a sphere, a nanorod, a nanocube, a nanoprism, and a nano shell.

Particle Diameter of Particle Containing Inorganic Material

In the present invention, the particle diameter of the particle containing the inorganic material is determined by measuring the diameter of an image of the particle containing the inorganic material taken with a transmission electron microscope (TEM). In the case where the image of the particle is not in the shape of a sphere, the lengths of the short axis and the long axis of the image of the particle containing the inorganic material are measured and an average value of them is taken as a diameter.

In the present embodiment, the particle diameter of the particle containing the inorganic material may be 1 nm to 950 nm, and even 15 nm to 500 nm. Moreover, 200 nm or less may be provided.

The molar absorption coefficient of the particle containing the inorganic material, according to the present embodiment, may be 10 M⁻¹cm⁻¹ or more, such as 10⁵ M⁻¹cm⁻¹ or more, and even 10⁷ M⁻¹cm⁻¹ or more, and further may be 10⁸ M⁻¹cm⁻¹ or more in a wavelength range of 600 nm to 1,300 nm.

Organic Dye

In the present embodiment, the organic dye is not specifically limited insofar as the organic dye absorbs light and emits an acoustic wave. Here, the light refers to ultraviolet light (an electromagnetic wave with a wavelength of 10 nm to 400 nm), visible light (an electromagnetic wave with a wavelength of 400 nm to 600 nm), near-infrared light (an electromagnetic wave with a wavelength of 600 nm to 1,300 nm), and the like. The organic dye in the present embodiment can be an organic dye which absorbs near-infrared light and emits an acoustic wave.

Examples of organic dyes in the present embodiment include cyanine based dyes, azine based dyes, acridine based dyes, beryllium based dyes, quinone based dyes, tetracycline based dyes, flavone based dyes, polyene based dyes, BODIPY (registered trademark) based dyes, Hilyte Fluor (registered trademark) based dyes, hemicyanine based dyes, rhodamine based dyes, streptocyanine based dyes, stilbene based dyes, styryl based dyes, merocyanine based dyes, polynuclear merocyanine based dyes, oxazole based dyes, oxonol based dyes, oxadiazole based dyes, arylidene based dyes, triphenylmethane based dyes, xanthene based dyes, azo based dyes, porphyrin based dyes, phthalocyanine based dyes, coumarine based dyes, naphthalimide based dyes, metal complex based dyes, and derivatives thereof. Among the above described dye groups, cyanine based dyes, e.g., Cy (registered trademark) 5.5 (produced by GE Healthcare UK) and Alexa Fluor (registered trademark) 750 (produced by Invitrogen), are suitable for use in a high moisture environment, e.g., in vivo, because an aromatic ring in the dye molecule has a sulfonic acid group so as to exhibit high water solubility.

Concrete examples of organic dyes according to the present embodiment include compounds represented by Formulae (1) to (4) described below.

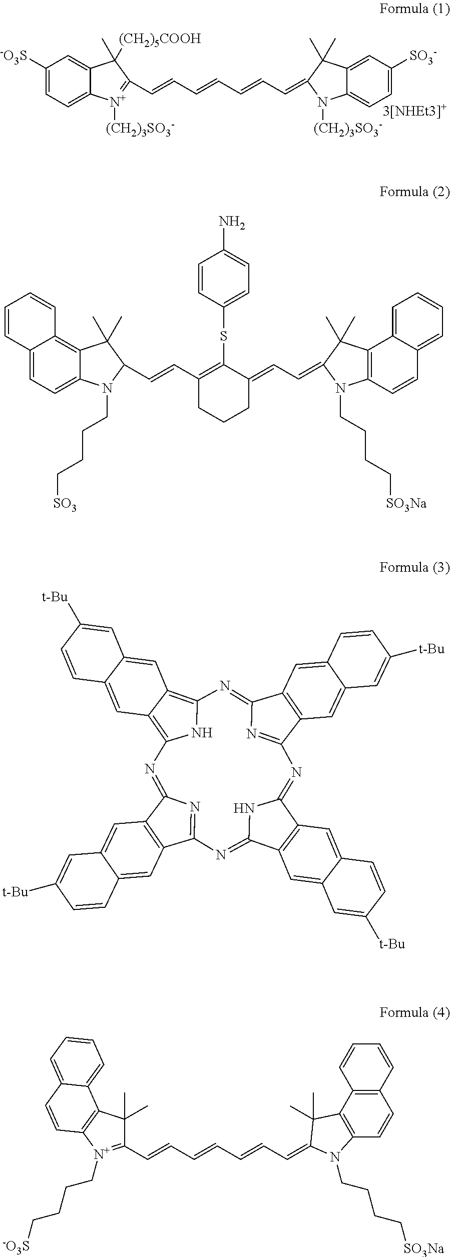

In the case where the organic dye is used as a second dye, the organic dye can be a polyfunctional dye having at least two reactive functional groups. Here, the term “reactive functional group” refers to a functional group which can be boded to a core particle under an appropriate condition and a functional group which can be bound to a functional group included in the antibody described later. Examples of reactive functional groups include a carboxyl group, an amino group, a maleimide group, a hydroxyl group, a thiol group, and a N-hydroxysuccinimide (NHS). Concrete examples of organic dyes having two reactive functional groups include Cy (registered trademark) 5.5 bisfunctional reactive dye (produced by GE Healthcare) represented by Formula (5) described below. The dye represented by Formula (5) described below has two maleimide groups serving as reactive functional groups. The organic dye represented by Formula (6) described below is Cy (registered trademark) 5.5 having two NHS groups serving as reactive functional groups. The organic dye represented by Formula (7) described below is a cyanine based dye having two carboxyl groups serving as reactive functional groups (hereafter may be referred to as Cypate).

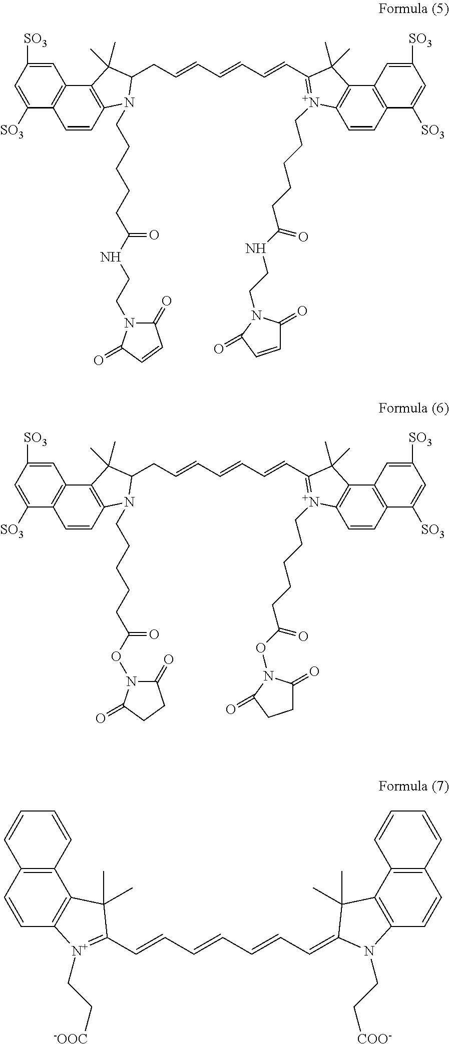

In the case where the organic dye has two reactive functional groups, those reactive functional groups can be different from each other. This is because when they are different, one reactive functional group included in the organic dye is bound to the core particle, and the other reactive functional group is not bound to the core particle easily so as to be bound easily to the antibody which is bound thereafter. For example, in the case where two reactive functional groups are included and they are the same, both the two functional groups may be bound to the core particle and, thereby, the antibody may not be bound to the organic dye.

Alternatively, a monofunctional dye, which is an organic dye having only one reactive functional group, may be converted to a polyfunctional dye through chemical modification and be used as the organic dye in the present embodiment. Example of monofunctional dyes include Cy (registered trademark) 5.5 monofunctional reactive dye (produced by GE Healthcare) represented by Formula (8) described below.

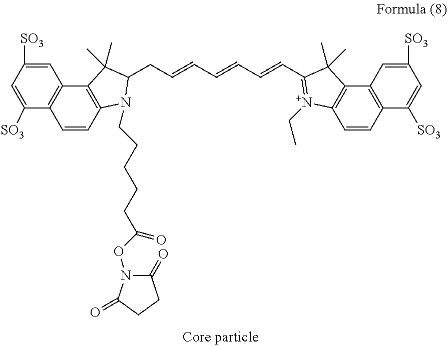

In the present embodiment, in the case where the core particle has at least one of the inorganic material and the organic dye, the inorganic material or the organic dye may be dispersed in an inorganic substance or an organic substance, or be coated with the inorganic substance or the organic substance. The core particle may be the inorganic material or the organic dye in itself.

Examples of the above described organic substances to disperse or cover the inorganic material or the organic dye include polysaccharide, protein, peptide, nucleic acids, synthetic polymers, liposomes, polymer micelles, polyion complexes, fatty acids, and surfactants.

Examples of the above described polysaccharide may include dextran, pullulan, mannan, amylopectin, chitosan, xyloglucan, hyaluronic acid, alginic acid, water-soluble cellulose, starch, agarose, carrageenan, and heparin. Furthermore, derivatives in which a functional group, e.g., an amino group, a hydroxyl group, a carboxyl group, or a maleimide group, is introduced in the polysaccharide may also be used.

Examples of the above described protein may include gelatin, collagen, albumin, and fibrin.

Examples of the above described synthesized polymers include polymers including an amino group, e.g., polyethyleneimines, polylysines, polyarginines, polyhistidines, polyallylamines, and polyamideamine dendrimers, polymers including a hydroxyl group, e.g., polyvinyl alcohols and polyethylene glycols, polymers including a carboxyl group, e.g., polyglutamic acids, polyasparagic acids, polymalic acids, polymethacrylic acids, polyacrylic acids, polyfumaric acids, and polymaleic acids, polymers containing an acid anhydride, e.g., polymaleic anhydrides, and polymers having biocompatibility, e.g., polylactic acids, polyglycolic acids, and polyethylene glycols.

Moreover, a copolymer including a monomer unit constituting the above described synthesized polymer and a monomer unit of other synthesized polymer may be used. Examples thereof include polylactic acid-glycolic acid copolymers.

Examples of phosphatide constituting the above described lypsomes include phosphatidylcoline, phosphatidylserine, phosphatidylinositol, phosphatidylglycelol, phosphatidylethanolamine, and sphingomyelin.

Examples of polymers to form the above described polymer micelles may include block copolymers having a hydrophilic segment formed from polyethylene glycol and a hydrophobic segment selected from the group consisting of polylactides, polylactide-glycolide copolymers, and poly-ε-caprolactones.

Examples of combinations of polymers to form the above described polyion complexes may include a polymer having a polycation segment selected from the group consisting of polyethyleneimines, polylysines, polyarginines, polyhistidines, and polyallylamines and a polymer having a polyanion segment selected from the group consisting of polyglutamic acids, polyasparagic acids, polymalic acids, polymethacrylic acids, and polyacrylic acids.

A copolymer having a monomer unit constituting a polymer to form the polyion complex and a monomer unit of another polymer may be used.

Examples of the above described fatty acids may include saturated fatty acids, e.g., lauric acid, myristic acid, palmitic acid, and stearic acid, unsaturated fatty acids, e.g., lauroleic acid, physeteric acid, myristoleic acid, palmitoleic acid, petroselinic acid, and oleic acid, and branched fatty acids, e.g., isolauric acid, isomyristic acid, isopalmitic acid, and isostearic acid.

Examples of the above described surfactants may include polyoxyethylene alkyl ethers, alkyl sulfonates, phosphatides, polyoxyethylene sorbitan based fatty acid esters.

Examples of the above described inorganic substances to disperse or cover the inorganic material or the organic dye may include silica, carbonates, and hydroxyapatite.

The above described organic substances and the inorganic substances to disperse or cover the inorganic material or the organic dye may be used alone or in an optional combination.

The organic substances and the inorganic substances to disperse or cover the inorganic material or the organic dye can have the above described reactive functional group. This is because the second dye is bound to the reactive functional group easily. Examples of the reactive functional groups include a carboxyl group, an amino group, a maleimide group, and a hydroxyl group.

As for a method for introducing this functional group into dextran, which is an example of the organic substance, to disperse or cover the inorganic material or the organic dye, a known chemical modification method may be used. For example, dextran particles are cross-linked with epichlorohydrin and, thereafter, are subjected to an ammonia treatment, so that dextran particles having an amino group are produced.

Binding Between Core Particle and Organic Dye

In the case where the core particle has the above described reactive functional group, the second dye may be bound to the core particle directly through a coupling reaction or the like.

Examples of direct binding include an amidation reaction. In the case where the core particle has a carboxyl group or an ester derivative thereof and the second dye has an amino group, for example, this reaction is effected through a condensation reaction of the carboxyl group or the ester derivative thereof and the amino group included in the second dye. In the case where the carboxyl group is amidated directly, a carbodiimide condensation agent, e.g., N,N′-dicyclohexylcarbodiimide or 1-ethyl-3-(3-dimethylaminopropyl) carbodiimide hydrochloride, may be used, and it is also possible to facilitate the amidation reaction by converting the carboxyl group to an activated ester in advance by using N-hydroxysuccinimide (NHS) or the like.

In addition, in the case where the core particle has a thiol group and the second dye has a maleimide group, a binding reaction between the thiol group and the maleimide group may be mentioned as an example. Regarding this reaction, an efficient selective binding reaction is effected in a region where a pH is neutral. The core particle binding to the second dye through this reaction may be washed and refined by an ultrafiltration method, gel filtration chromatography, or the like. In the case where the core particle is a ferromagnetic material, for example, in the case where the core particle contains iron oxide, washing and refining may be performed by a magnetic separation method through the use of a permanent magnet. Meanwhile, in the case where the core particle is very small and exhibits superparamagnetism, the core particle may be washed and refined by using a magnetic column under a high gradient magnetic field.

The method for binding the core particle and the second dye and the type of the functional group included in the second dye are not limited to those described above, and a person skilled in the art may select appropriately from available various binding methods and known functional groups.

As described above, the second dye absorbs light and, thereby, emits an acoustic wave. Therefore, it is better that the second dye bound to the core particle becomes higher in number.

Antibody

In the present embodiment, the antibody is a generic name for proteins of an immunoglobulin family which are induced by an immune system in response to a specific antigen or a substance and refers to a substance which recognize a specific target molecule and which can be bound to the target molecule specifically. In the present invention, the term “to bind specifically” is defined as to have a dissociation constant KD (the binding affinity becomes higher as the value becomes smaller) from the target molecule of 1 μM or less.

In the present embodiment, the antibody may be a mouse antibody, a human antibody, a humanized antibody, or a chimeric antibody, or be derived from other species. Furthermore, the antibody may be either a monoclonal antibody or a polyclonal antibody.

The antibody in the present embodiment may be a whole antibody or an antibody fragment. The antibody fragment refers to a part of an antibody and can be bound specifically to a target molecule. Examples of antibody fragments include a Fab fragment (hereafter may be referred to as “Fab”), a Fab′ fragment (hereafter may be referred to as “Fab′”), F(ab′)₂, a single heavy chain variable (VH) region, a single light chain variable (VL) region, a composite of VH and VL, a camelised VH domain, a peptide containing a complementality determining region (CDR) of an antibody, and a single chain variable fragment (scfv) in which the heavy chain variable region and the single light chain variable region are joined. The single chain variable fragment can be a humanized single chain variable fragment. The single chain variable fragment is produced in accordance with various antigens inexpensively and simply. The single chain variable fragment has a small molecular weight as compared with that of the whole antibody and the like and, therefore, is possible to bind antibodies higher in number to the particle containing the inorganic material. Furthermore, it is believed that the single chain variable fragment has no Fc region (constant region) of an antibody and, therefore, has low antigenicity. Consequently, the single chain variable fragment can be the antibody of the composite particle according to the present embodiment.

Target Molecule

The composite particle according to the present embodiment is used for, for example, photoacoustic imaging to detect a pathological tissue, e.g., a tumor. Therefore, the above described target molecule is, for example, a molecule expressed specifically in a lesion resion, in particular a molecule expressed specifically in a tumor region. Concrete examples include tumor antigens, receptors, membrane proteins of cell surfaces, proteolytic enzymes, and cytokine.

Concrete examples of the above described tumor antigens include Vascular Endothelial Growth Factor (VEGF) family, Vascular Endothelial Growth Factor Receptor (VEGFR) family, Prostate Specific Antigen (PSA), Carcinoembryonic Antigen (CEA), Matrix Metalloproteinase (MMP) family, Epidermal Growth Factor Receptor (EGFR) family, Eptdermal Growth Factor (EGF), Integrin family, Type 1 Insulin-Like Growth Factor Receptor: IGF-1R), CD 184 Antigen (CXC Chemokine Receptor 4: CXCR4), and Placental Growth Factor: PIGF). In particular, Human Epidermal Growth Factor Receptor 2 (HER2) of EGFR family can be mentioned. HER2 may be referred to as ErbB2, c-Erb-B2, and p185^{HER2}. HER2 is one of tyrosine kinase receptors. HER2 is a substance (protein) expressed excessively through gene amplification with respect to adenocarcinomata, e.g., breast cancer, prostatic carcinoma, stomach cancer, ovarian cancer, and pulmonary cancer. Therefore, in the case where an antibody which is bound specifically to HER2 is used as the antibody included in the composite particle according to the present embodiment, a contrast agent for photoacoustic imaging, which can detect specifically a tumor region of the above described cancer, is produced. In this regard, as for the antibody which is bound specifically to HER2, Herceptin (registered trademark) (produced by CHUGAI PHARMACEUTICAL CO., LTD.) is mentioned.

The composite particle according to the present embodiment may be used as the contrast agent for photoacoustic imaging and, in addition, be used as contrast agents for magnetic resonance imaging (MRI) and fluorescence imaging. Furthermore, in the case where the particle containing iron oxide has ferromagneticity or paramagnetism, the particle may be used for magnetic separation or refining of HER2 taking the advantage of the property to bind specifically to HER2.

Regarding the antibody in the present embodiment, for example, the antibody may be produced appropriately by a known antibody manufacturing method, wherein the above described antigen or a partial peptide thereof is used as an immunogen. Furthermore, the above described single chain variable fragment may be obtained as a recombinant protein from the gene arrangement information of the resulting antibody by a gene recombination method. Moreover, a commercially available antibody may be used.

Binding Between Second Dye and Antibody

In the case where the second dye has the above described reactive functional group, the second dye may be bound directly to the antibody through a coupling reaction or the like.

Examples of direct binding include an amidation reaction. This reaction is effected through, for example, a condensation reaction between a carboxyl group or an ester derivative thereof included in the core particle and an amino group included in the antibody. In the case where the carboxyl group is amidated directly, a carbodiimide condensation agent, e.g., N,N′-dicyclohexylcarbodiimide or 1-ethyl-3-(3-dimethylaminopropyl) carbodiimide hydrochloride, may be used, and it is possible to facilitate the amidation reaction by converting the carboxyl group to an activated ester in advance by using N-hydroxysuccinimide (NHS) or the like.

In addition, in the case where the second dye has a thiol group and the antibody has a maleimide group, a binding between the thiol group and the maleimide group may be mentioned. Regarding this reaction, an efficient selective binding reaction is effected in a region where a pH is neutral. The composite particle, according to the present embodiment, produced by binding the antibody to the second dye, which is bound to the core particle, may be washed and refined by an ultrafiltration method, gel filtration chromatography, or the like. In the case where the core particle is a ferromagnetic material, for example, in the case where the core particle contains iron oxide, washing and refining may be performed by a magnetic separation method through the use of a permanent magnet. Meanwhile, in the case where the core particle is very small and exhibits superparamagnetism, the composite particle may be washed and refined by using a magnetic column under a high gradient magnetic field.

The method for binding the second dye and the antibody and the type of the functional group included in the core particle are not limited to those described above, and a person skilled in the art may select appropriately from available various binding methods and known functional groups.

Particle Diameter of Composite Particle

In the present embodiment, the particle diameter of the composite particle refers to a hydrodynamic diameter measured by a dynamic light scattering (DLS) method through the use of a dynamic light scattering analyzer (DLS-8000, produced by OTSUKA ELECTRONICS CO., LTD.). The particle diameter of the composite particle according to the present embodiment may be adjusted to an optimum particle diameter in accordance with the presence region of the above described target molecule. However, the particle diameter may be 1 nm to 1,000 nm, and even 15 nm to 500 nm.

In the case where the particle diameter of the composite particle is 1,000 nm or less, the contrast agent is accumulated in a tumor region to a greater extent as compared with that in a normal region of a living body on the basis of an enhanced permeability and retention (EPR) effect. As a result, when the living body is irradiated with the light after the composite particle is administrated to the living body, the photoacoustic signal produced from a tumor region becomes larger than the photoacoustic signal produced from a normal region. Therefore, the tumor region is detected specifically by adjusting the particle diameter of the composite particle to 1,000 nm or less. The particle diameter of the composite particle may be 200 nm or less. The reason for this is believed to be that in the case where the particle diameter of the composite particle is 200 nm or less, the composite particle is not easily taken into a macrophage in a blood and, thereby the retentivity in the blood increases.

### Second Embodiment

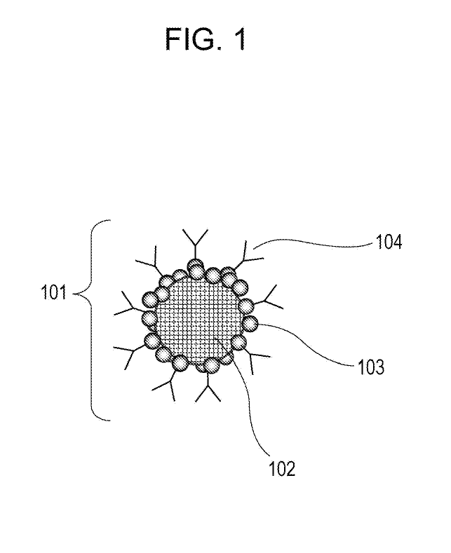

A composite particle according to a second embodiment of the present invention will be described with reference to FIG. 1. Regarding the individual constituents of the composite particle according to the present embodiment, explanations of those described in the first embodiment will not be provided here.

The composite particle 101 according to the present embodiment is formed from a particle 102 containing an inorganic material, organic dyes 103 bound to the above described particle, and antibodies 104. Then, it is a feature that the antibody 104 is bound to the organic dye 103.

As described above, the organic dye 103 is bound to a binding site, to which the organic dye can be bound, of the particle containing iron oxide. The antibody 104 may be bound to a part of or the whole organic dye 103 bound to the particle 102. In the case where the organic dye 103 has a plurality of binding sites, to which the antibody 104 can be bound, a plurality of antibodies 104 may be bound to one organic dye. The composite particle includes both the organic dye 103 and the antibody 104 high in number by employing such a form. Regarding the resulting contrast agent, when the composite particle is irradiated with light, a large acoustic wave can be emitted and an antigen region can be detected more accurately.

In the case where the organic dye 103 is not bound to all binding sites, to which the organic dye can be bound, of the particle including the iron oxide particle, the antibody 104 may be bound to the binding sites, to which the organic dye 103 is not bound. Furthermore, another antibody may be bound to the antibody 104.

The composite particle, in which the organic dye is bound to the particle containing the inorganic material and the antibody (not emitting an acoustic wave) is bound to the organic dye, emits an acoustic wave larger than that of the composite particle, in which the organic dye is bound to the particle containing the inorganic material. The reason for this is believed to be that in the case where the antibody is bound to the organic dye, the excitation energy obtained by irradiating the organic dye with the light is not emitted as fluorescence easily. That is, when the antibody is bound to the organic dye, at least a part of the excitation energy of the organic dye is converted to vibration and rotation energy of the antibody or thermal energy of a surrounding medium. It is believed that at least a part of the vibration and rotation energy of the antibody is transferred to the inorganic material and the surrounding medium and is consumed as thermal energy, so as to be emitted as an acoustic wave. As a result, it is believed that in the case where the antibody is bound to the organic dye, a large acoustic wave is generated as compared with the case where the antibody is not bound.

Method for Manufacturing Composite Particle

A method for manufacturing the composite particle according to the present embodiment includes the steps of binding the second dye to a particle containing the first dye having a molar absorption coefficient of 10 M⁻¹cm⁻¹ or more at least one wavelength selected within the range of 600 nm to 1,300 nm and binding the antibody to the above described second dye.

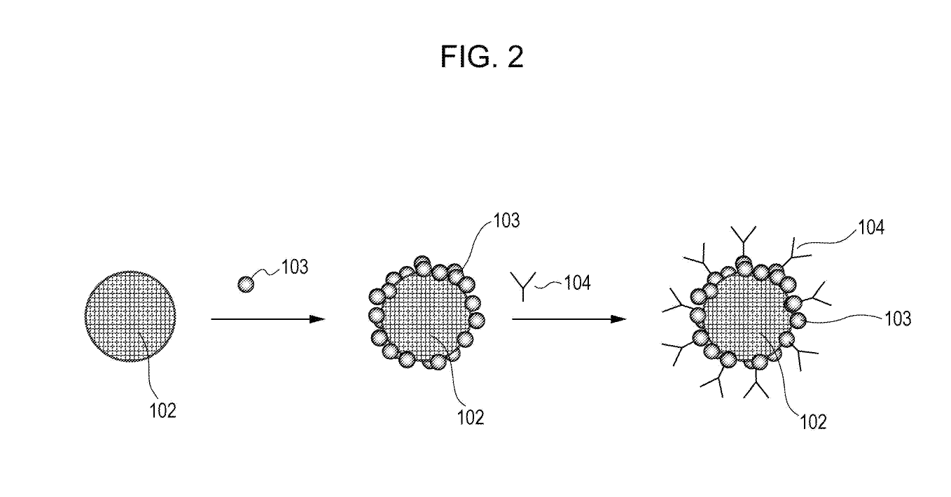

An example of a method for manufacturing the composite particle according to the present embodiment will be described with reference to FIG. 2. Initially, the organic dye 103 is bound to the particle 102 containing the inorganic material. Subsequently, the antibody 104 is bound to the organic dye bound to the particle containing the inorganic material. The composite material may be prepared as described above. In this regard, after the antibody 104 is bound to the organic dye 103, the composite material may be prepared by binding the organic dye binding to the antibody to the particle containing the inorganic material.

Contrast Agent for Photoacoustic Imaging

The composite particle according to the present embodiment may be used as a contrast agent for photoacoustic imaging because the light is absorbed and, thereby, an acoustic wave is emitted.

Here, the term “contrast agent” in the present invention is defined mainly as a substance which is allowed to be present in a specimen and which can generate contrast difference between a tissue or a molecule to be observed and a tissue or a molecule surrounding it, so as to improve the detection sensitivity of the morphological information or position information of the tissue or the molecule to be observed. Here, the term “photoacoustic imaging” refers to imaging of the above described target molecule with a photoacoustic apparatus or the like. The term “contrast agent for photoacoustic imaging” refers to a contrast agent which can be used for the photoacoustic imaging concerned.

Dispersion Medium

The contrast agent according to the present embodiment includes the above described composite particle and the dispersion medium according to the present embodiment. The above described dispersion medium is a liquid substance to disperse the composite particles according to the present embodiment, and examples thereof include physiological saline and distilled water for injection. Regarding the contrast agent according to the present embodiment, the above described composite particles according to the present embodiment may be dispersed in this dispersion medium in advance or the composite particles according to the present embodiment and the dispersion medium may be prepared in kit form and be used by dispersing the composite particles into the dispersion medium before being administrated to a living body. The contrast agent according to the present embodiment may contain, optionally, pharmacologically allowable additives besides the composite particles according to the present embodiment and the dispersion medium.

Photoacoustic Imaging Method

A photoacoustic imaging method according to the present embodiment includes the steps of irradiating a specimen, which has administrated with the above described contrast agent for photoacoustic imaging, with the light within a wavelength region of 600 nm to 1,300 nm and detecting an acoustic wave emitted from the contrast agent present in the above described specimen.

An example of the photoacoustic imaging method according to the present embodiment is as described below. That is, the contrast agent according to the present embodiment is administrated to a specimen or is added to a sample, e.g., an organ, obtained from the above described specimen. In this regard, the above described specimen may be mammals, e.g., laboratory animals or pets other than a human, or others and is not specifically limited. Examples of samples in the above described specimen or samples obtained from the specimen include organs, tissues, tissue slices, cells, and cell solutions. After the contrast agent according to the present embodiment is administrated or added, the above described specimen or the like is irradiated with the laser pulse light in the near-infrared wavelength region.

Then, a photoacoustic signal (acoustic wave) from the contrast agent according to the present embodiment is detected with an acoustic wave detector, for example, a piezoelectric transducer, so as to be converted to an electric signal. On the basis of the electric signal obtained from this acoustic wave detector, the position and the size of an absorber in the above described specimen or the like or the optical characteristic value distributions of light absorption coefficient and the like are calculated. For example, in the case where the above described photoacoustic signal more than or equal to a reference threshold value is detected, it is estimated that the target molecule or a region which produces the target molecule is present in the specimen or it is estimated that the target molecule is present in the sample or a region which produces the target molecule is present in the specimen serving as an origin of the sample.

### EXAMPLES

Concrete reagents and reaction conditions used in production of the composite particles in the following examples are mentioned, although these reagents and reaction conditions may be modified, and such modifications are included in the scope of the present invention. Therefore, the following examples are to enhance understanding of the present invention and do not limit the scope of the present invention.

### Production of Particle Containing Inorganic Material

### Synthesis Example 1

Binding Between Polyfunctional Dye and Particle Including Iron Oxide Particle (1)

As for the organic dye, Cy 5.5 bisfunctional reactive dye (produced by GE Healthcare) (hereafter may be referred to as Dye A) serving as a polyfunctional dye represented by Formula (5) described above was used. A reaction between nanomag (registered trademark)-D-spio (produced by Micromod, average particle diameter 50 nm) (hereafter may be referred to as NP), which is a particle including the iron oxide particle and having Dye A and an amino group on the surface, and Cy 5.5 was effected at room temperature for 1 hour. The reaction solvent was a carbonate buffer and the pH was specified to be 8. The reaction molar ratio (fed Dye A/particle including iron oxide particle) was specified to be 0, 1,600, or 8,330. Here, the term “fed” refers to “added to a reaction system” and the term “reaction molar ratio” refers to “ratio of the molarity of Dye A added to the reaction system to the molarity of particle including iron oxide particle”.

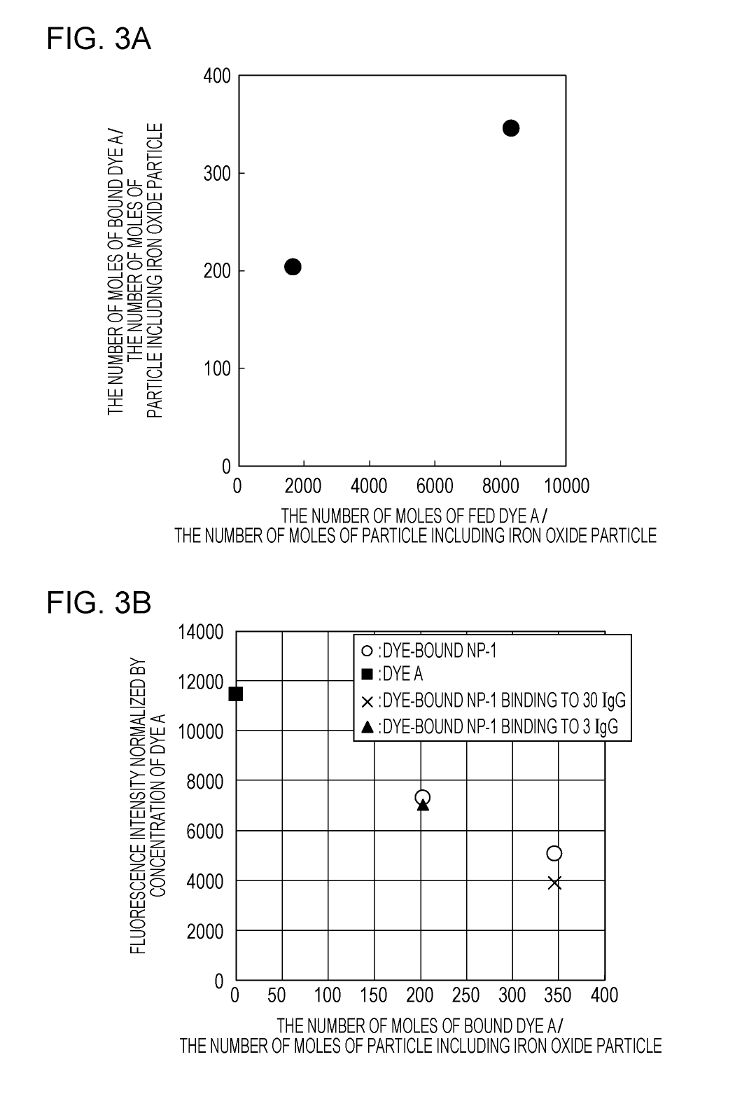

After the reaction, Dye A not bound to NP was removed through ultrafiltration by using Amicon Ultra-4 (produced by Nihon Millipore K.K.) having a pore size of 100 kDa, so as to obtain NP to which Dye A was bound (hereafter may be referred to as dye-bound NP-1). The amount of binding of Dye A to NP was calculated from the absorbance at 675 nm of the resulting dye-bound NP-1. In this regard, the molar absorption coefficient at 675 nm of the Dye A was 2.5×10⁵ M⁻¹cm⁻¹ or more. FIG. 3A shows the relationship between the number of moles of fed Dye A/the number of moles of particle including iron oxide particle and the number of moles of bound Dye A/the number of moles of particle including iron oxide particle. It was able to be ascertained that as the number of moles of fed Dye A/the number of moles of particle including iron oxide particle increased, the number of moles of bound Dye A/the number of moles of particle including iron oxide particle increased.

Table 1 shows the relationship between the number of moles of bound Dye A/the number of moles of particle including iron oxide particle and the molar absorption coefficient (M⁻¹cm⁻¹) of the particle binding to Dye A and including the iron oxide particle (dye-bound NP-1). It is clear that the particle modified with 346 Dyes A had a molar absorption coefficient 6.6 times the molar absorption coefficient of the particle binding to no Dye A.

TABLE 1

| | Molar absorption coefficient of |
|---|---|
| The number of moles of bound Dye | particle binding to Dye A and |
| A/the number of moles of particle | including iron oxide particle |
| including iron oxide particle | (M⁻¹cm⁻¹) |
| 0 | 1.3 × 10⁷ |
| 203 | 5.0 × 10⁷ |
| 346 | 8.6 × 10⁷ |

### Example 1

Preparation of Composite Particle (1)

In the present example, the composite particle was prepared, that is, an antibody was bound to the dye-bound NP-1. Initially, 20 mg of 1-ethyl-3-(3-dimethylaminopropyl)carbodiimide hydrochloride and 24 mg of N-hydroxysuccinimide were dissolved into 1 ml of 0.5 M β-morpholinoethnesulfonic acid buffer solution (pH=6.3). Subsequently, the resulting solution was added to 1 ml of particle suspension liquid containing the dye-bound NP-1. The resulting particle suspension liquid was agitated at room temperature for 1 hour. Thereafter, PD-10 desalting column (produced by GE Healthcare Bioscience) was used and, thereby, a liquid in which a carboxyl group included in Dye A was activated and a low molecular reagent were separated. As for a developing solvent, sodium carbonate buffer (pH 8.0) was used, and buffer exchange was performed at the same time. Then, Herceptin (registered trademark) (produced by CHUGAI PHARMACEUTICAL CO., LTD.) (hereafter may be referred to as IgG) serving as an anti-HER2 antibody was added as an antibody to the resulting particle suspension liquid. The reaction molar ratio (IgG/dye-bound NP-1) was specified to be 100. After the particle suspension liquid was agitated at room temperature for 4 hours, a 1 M aqueous solution of glycine was added in such a way that the glycine final concentration became 1 mM, and agitation was performed at room temperature for 30 minutes. The resulting composite particle, that is, the dye-bound NP-1 containing IgG, was refined through gel filtration chromatography (200GL10/300 column of Superdex, produced by GE Healthcare Bioscience). As for the developing solvent, a phosphate buffered saline (PBS, pH=7.4) was used. The dye-bound NP-1 binding to IgG was eluted as a void volume fraction and this was recovered. In this regard, the amount of binding of IgG to the dye-bound NP-1 was calculated by quantifying the concentration of unreacted IgG from the peak area derived from the unreacted IgG. The number of binding of IgG to the dye-bound NP-1 was 30 per dye-bound NP-1. In the case where IgG was bound directly to the dye-bound NP-1 without the above described step to activate the carboxyl group, the number of binding of IgG to the dye-bound NP-1 was 3 per dye-bound NP-1. As is clear from this result, a part of NHS groups of Dye A bound to the particle including the iron oxide particle remained during binding to IgG, although most of them were deactivated through hydrolysis.

Measurement of Fluorescence Intensity of Composite Particle

The composite particle was subjected to fluorescence spectrum observation, and the fluorescence intensity was measured. The excitation wavelength was specified to be 675 nm, and the fluorescence intensity was measured at a fluorescence wavelength of 694 nm. For the purpose of comparison, the fluorescence intensity of the Dye A alone was measured. The results are shown in FIG. 3B. In FIG. 3B, the relationship between the fluorescence intensity normalized by the concentration of Dye A and the number of moles of bound Dye A/the number of moles of particle including iron oxide particle. The fluorescence intensity from the dye-bound NP-1 decreased to 44% to 64% of the fluorescence intensity of Dye A alone and, therefore, it was made clear that as the number of moles of bound Dye A/the number of moles of particle including iron oxide particle increased, the fluorescence intensity decreased. It is believed that this is mainly because of concentration quenching between one Dye A and another Dye A. Furthermore, it was made clear that the fluorescence intensity from the dye-bound NP-1 binding to IgG was lower than the fluorescence intensity of NP binding to no IgG. It is believed that this is mainly because at least a part of the excitation energy of Dye A was converted to the vibration and rotation energy of IgG or thermal energy of a surrounding medium. Moreover, it was made clear that the fluorescence intensity normalized by the concentration of Dye A of the dye-bound NP-1 binding to 30 IgG (composite particle including 30 IgG) was low as compared with that of the dye-bound NP-1 binding to 3 IgG (composite particle including 3 IgG).

### Reference Example 1

Binding Between Antibody and Particle Including Iron Oxide Particle and Binding to Monofunctional Dye

As for a monofunctional dye, Cy 5.5 monofunctional reactive dye (produced by GE Healthcare) was used. This monofunctional dye and NP described above were bound following the method in the above described example (hereafter may be referred to as monofunctional dye-bound NP). The reaction molar ratio (monofunctional dye-bound NP/particle including iron oxide particle) was specified to be 1,600. The number of moles of monofunctional dye/the number of moles of NP was 143 on the basis of the absorbance at 675 nm of the resulting monofunctional dye-bound NP. A cross-linking molecule having two NHS groups was used in order to bind IgG to the monofunctional dye-bound NP. Concretely, bis(sulfosuccinimidyl) suberate (BS3, produced by Thermo Fisher Scientific K.K.) was added to the particle suspension liquid. The resulting particle suspension liquid was agitated at room temperature for 1 hour. Thereafter, PD-10 desalting column (produced by GE Healthcare Bioscience) was used and, thereby, the monofunctional dye-bound NP, in which a surface amino group was NHS-esterified, and an unreacted BS3 were separated. Then, IgG was reacted in the same manner as that of the binding reaction of IgG in Example 1 described above.

As a result, it was not ascertained that IgG was bound. Consequently, it was shown that binding of the antibody to the surface of the monofunctional dye-bound NP was difficult.

### Synthesis Example 2

Binding Between Polyfunctional Dye and Particle Including Iron Oxide Particle

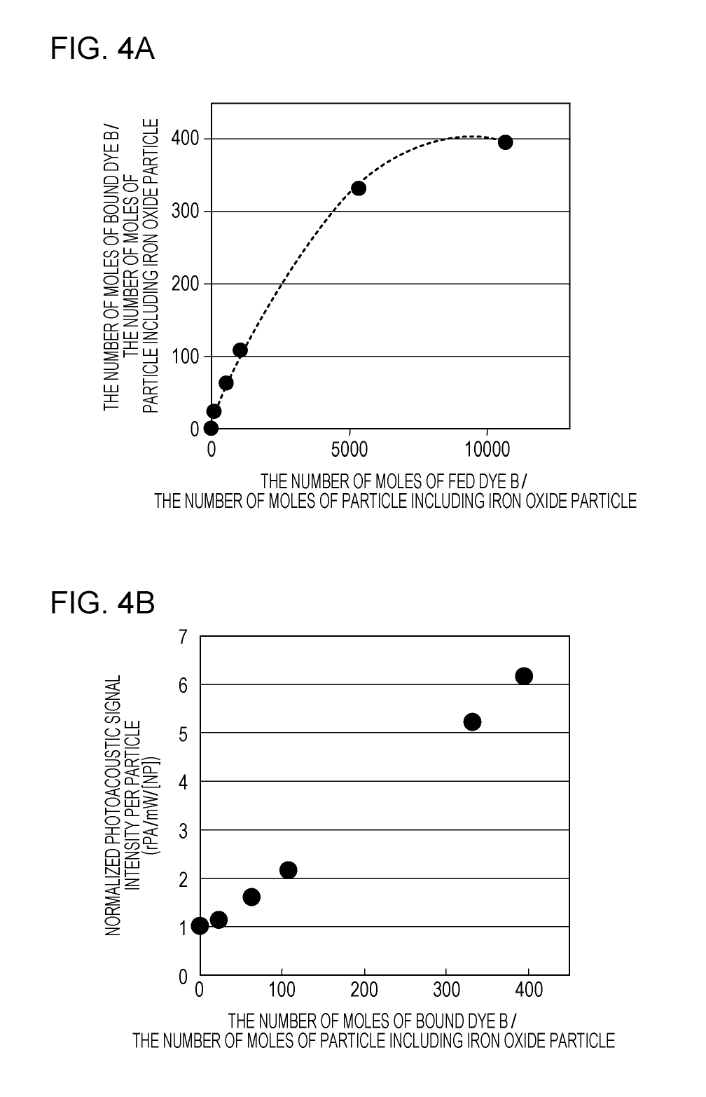

In the present synthesis example, as for the organic dye, Hilyte Fluor (registered trademark) 750 Bis-NHS ester, isomer II TEA salt (produced by ANASPEC, hereafter may be referred to as Dye B) serving as a polyfunctional dye was used. A reaction between Dye B and NP described above was effected at room temperature for 1 hour. The reaction solvent was a carbonate buffer and the pH was specified to be 8. Synthesis reactions were effected while the reaction molar ratio (the number of moles of fed Dye B/the number of moles of particle including iron oxide particle) was specified to be 0, 86, 535, 1,069, 5,347, and 10,694. After the reaction, Dye B not bound to NP was removed through ultrafiltration by using Amicon Ultra-4 (produced by Nihon Millipore K.K.) having a pore size of 100 kDa, so as to obtain NP to which Dye B was bound (hereafter may be referred to as dye-bound NP-2). The amount of binding of Dye B to NP was calculated from the absorbance at 750 nm of the resulting dye-bound NP-2. In this regard, the molar absorption coefficient at 750 nm of the Dye B was 2.5×10⁵ M⁻¹cm⁻¹. Regarding the resulting dye-bound NP-2, FIG. 4A shows the relationship between the number of moles of fed Dye B/the number of moles of particle including iron oxide particle and the number of moles of bound Dye B/the number of moles of particle including iron oxide particle. It was able to be ascertained that as the number of moles of fed Dye B/the number of moles of particle including iron oxide particle increased, the number of moles of bound Dye B/the number of moles of particle including iron oxide particle increased. Table 2 shows the relationship between the number of moles of bound Dye B/the number of moles of particle including iron oxide particle and the molar absorption coefficient of the dye-bound NP-2. It was made clear that in the case where the number of moles of bound Dye B/the number of moles of particle including iron oxide particle was 395 (hereafter may be abbreviated as NP-2-395), the molar absorption coefficient was 9.2 times the molar absorption coefficient in the case where Dye B was not bound.

TABLE 2

| | Molar absorption coefficient of |
|---|---|
| The number of moles of bound Dye | particle binding to Dye B and |
| B/the number of moles of particle | including iron oxide particle |
| including iron oxide particle | (M⁻¹cm⁻¹) |
| 0 | 1.3 × 10⁷ |
| 23 | 1.7 × 10⁷ |
| 63 | 2.7 × 10⁷ |
| 108 | 4.1 × 10⁷ |
| 332 | 1.0 × 10⁸ |
| 395 | 1.2 × 10⁸ |

Measurement of Photoacoustic Signal of Polyfunctional Dye-Bound NP-2

The intensity of the photoacoustic signal of the dye-bound NP-2 obtained as described above was measured. For the purpose of comparison, NP binding to no Dye B was measured in the same manner.

Regarding the measurement of the photoacoustic signal, a sample was irradiated with the pulse laser light, and a photoacoustic signal from the sample was detected by using a piezoelectric element and was amplified with a high-speed preamplifier. Thereafter, the photoacoustic signal was got with a digital oscilloscope. The concrete condition was as described below. As for a light source, a titanium sapphire laser (produced by Lotis) was used. As for the condition, the wavelength was 750 nm, the energy density was 12 mJ/cm², the pulse width was 20 nanoseconds, and the pulse repetition was 10 Hz. As for an ultrasonic transducer, Model V303 (produced by Panametrics-NDT) was used. As for the condition, the central band was 1 MHz, the element size was Φ0.5, the measurement distance was 25 mm (Non-focus), and an amplifier was +30 dB (Ultrasonic Preamplifier Model 5682 produced by Olympus Corporation). A measurement container was a polystyrene cuvette having an optical path length of 0.1 cm and a sample capacity of about 200 μl. As for a solvent, PBS was used. As for a measuring device, DPO4104 (produced by Tektronix) was used, and the measurement was performed under the condition of Trigger: detection of photoacoustic light with a photodiode and Data acquisition: average of 128 times (128 pulses).

The results are shown in FIG. 4B. FIG. 4B shows the relationship between the relative photoacoustic signal intensity normalized by the particle concentration and the number of moles of bound Dye B/the number of moles of particle including iron oxide particle. In FIG. 4B, the number of moles of bound Dye B/the number of moles of particle including iron oxide particle of 0 refers to NP binding to no Dye B, and the photoacoustic signal intensity of this NP was specified to be 1. As is clear from FIG. 4B, the photoacoustic signal increased as the number of Dye B increased. It was made clear that the photoacoustic signal intensity measured from NP-2-395 having the largest number of bound Dye B was about 6 times the photoacoustic signal intensity of NP binding to no Dye B.

### Example 2

Preparation of Composite Particle (2)

In the present example, the composite particle was prepared by binding IgG described above to NP-2-395 prepared as described above. Concretely, 20 mg of 1-ethyl-3-(3-dimethylaminopropyl)carbodiimide hydrochloride and 24 mg of N-hydroxysuccinimide were dissolved into 1 ml of 0.5 M β-morpholinoethnesulfonic acid buffer solution (pH=6.3). Subsequently, the resulting solution was added to 1 ml of NP-2-395 suspension liquid. The resulting NP-2-395 suspension liquid was agitated at room temperature for 1 hour. Thereafter, PD-10 desalting column (produced by GE Healthcare Bioscience) was used and, thereby, NP-2-395 in which a carboxyl group included in Dye B was activated and a low molecular reagent were separated. As for a developing solvent, sodium carbonate buffer (pH 8.0) was used, and buffer exchange was performed at the same time. Then, IgG described above was added to the resulting NP-2-395 suspension liquid. The reaction molar ratio (the number of moles of IgG/the number of moles of dye-bound NP-2) was specified to be 100. After the NP-2-395 suspension liquid was agitated at room temperature for 4 hours, a 1 M aqueous solution of glycine was added in such a way that the glycine final concentration became 1 mM, and agitation was performed at room temperature for 30 minutes. The resulting NP-2-395 containing IgG (hereafter may be abbreviated as NP-2-395-IgG) was refined through gel filtration chromatography (200GL10/300 column of Superdex, produced by GE Healthcare Bioscience). As for the developing solvent, a phosphate buffered saline (PBS, pH=7.4) was used. NP-2-395-IgG was eluted as a void volume fraction and this was recovered. In this regard, the number of binding of IgG to NP-2-395-IgG was calculated by quantifying the concentration of unreacted antibody from the peak area derived from the unreacted antibody. The number of binding of IgG to the dye-bound NP-2 was 25 per dye-bound NP-2.

Measurement of Fluorescence Intensity of NP-2-395-IgG

The resulting NP-2-395-IgG was subjected to fluorescence spectrum observation, and the fluorescence intensity was measured. The excitation wavelength was specified to be 757 nm, and the intensity was measured at a fluorescence wavelength of 780 nm. For the purpose of comparison, the fluorescence intensity of NP-2-395 before binding of IgG was measured. As a result, it was made clear that when a comparison was made at the same particle concentration, the fluorescence intensity from NP-2-395-IgG decreased to 87% of the fluorescence intensity of NP-2-395. It is believed that this is mainly because of concentration quenching between dyes. Furthermore, the fluorescence intensity from the dye-bound NP-2 binding to IgG was lower than the fluorescence intensity of the dye-bound NP-2 boning to no IgG. It is believed that this is mainly because at least a part of the excitation energy of Dye B was converted to the vibration and rotation energy of IgG or thermal energy of a surrounding medium.

Measurement of Photoacoustic Signal of NP-2-395-IgG

The photoacoustic signal of the resulting NP-2-395-IgG was measured. For the purpose of comparison, ferucarbotran (trade name: Resovist (registered trademark) note, produced by Nihon Shering K.K.) which is commercially available iron oxide nanoparticles, NP binding to no Dye B, and NP-2-395 binding to no IgG were measured in the same manner. The photoacoustic signal was measured in the same manner as that in Synthesis example 2.

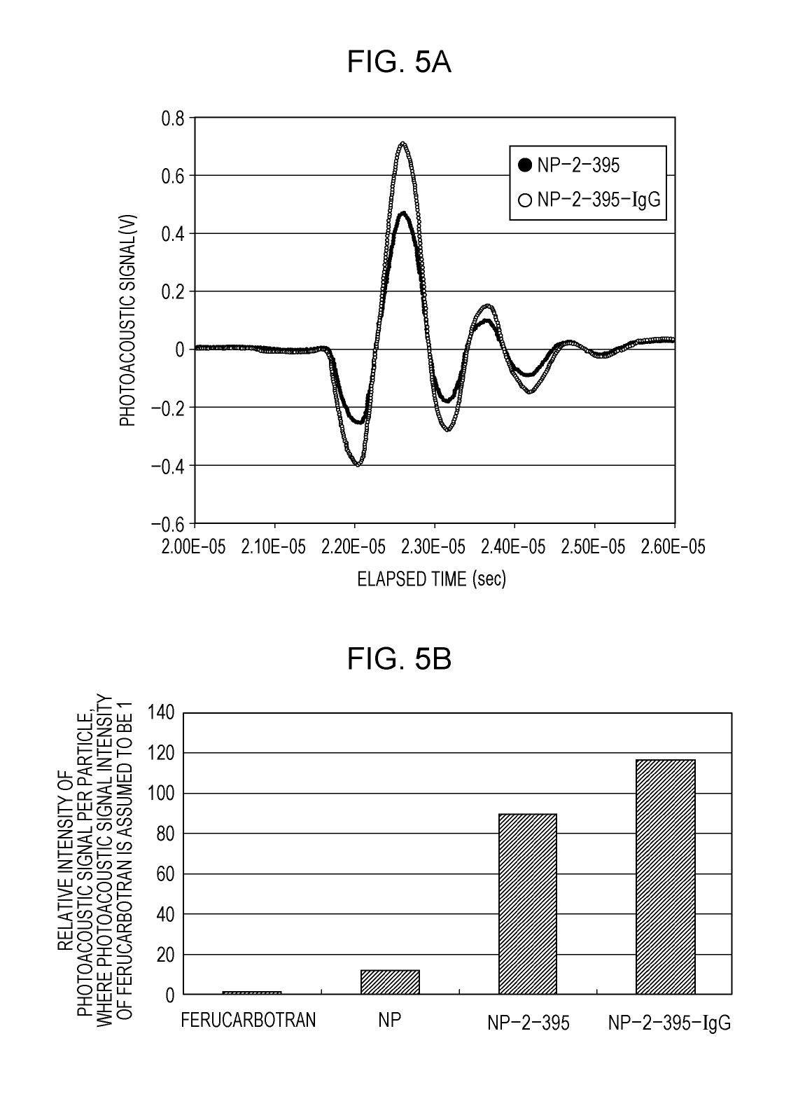

The results of measurement of the above described photoacoustic signal intensity are shown in FIGS. 5A and 5B. FIG. 5A shows photoacoustic signal waveforms derived from NP-2-395 (black circle) and NP-2-395-IgG (white circle) as typical examples. It is considered that peaks detected after time lag are affected by reflection in a cell and the like and, therefore, only the initial peak is effective as a photoacoustic signal from the sample. FIG. 5B shows the photoacoustic signal intensities per unit particle concentration of NP, NP-2-395, and NP-2-395-IgG, where the photoacoustic signal intensity of ferucarbotran described above was specified to be 1. The value of photoacoustic signal intensity from NP-2-395-IgG was 1.3 times larger than the value of photoacoustic signal intensity of NP-2-395. The reason for this increase in photoacoustic signal intensity is considered as described below. That is, it is believed that at least a part of the excitation energy of the organic dye is converted to vibration and rotation energy of IgG or thermal energy of a surrounding medium, at least a part of the vibration and rotation energy of IgG is transferred to the inorganic material and the surrounding medium so as to be consumed as thermal energy and, as a result, acoustic wave is emitted. Consequently, it is believed that in the case where IgG is bound to the organic dye, a large acoustic wave is generated as compared with the case where IgG is not bound. It is believed that the above described results of photoacoustic signal intensity measurement of NP-2-395-IgG supports the above described mechanism. Alternatively, as another mechanism, it is also considered that as a result of coverage of particle due to binding of IgG, a heat-confining effect of the particle increases and the photoacoustic signal intensity increases.

As is clear from FIG. 5B, the photoacoustic signal intensity of NP-2-395-IgG was about 120 times that of ferucarbotran.

From the above described results, it is believed that the composite particle according to the present embodiment serves as a contrast agent for photoacoustic imaging, wherein a large acoustic signal is produced, that is, a large photoacoustic signal is emitted.

### Example 3

Preparation of Composite Particle in which IgG is Bound to Polyfunctional Dye

Dye B serving as a polyfunctional dye was bound to NP in the same manner as that in Synthesis example 2 described above. Here, the reaction was effected while the reaction molar ratio (the number of moles of fed Dye B/the number of moles of particle including iron oxide particle) was specified to be 500. Then, a composite particle was prepared by binding IgG to the dye of the resulting dye-bound NP (hereafter abbreviated as NP-3) in the same manner as that in Example 2 (Preparation of composite particle (2)) described above. The amount of binding of dye, the amount of binding of antibody, the relative fluorescence intensity (relative value of fluorescence intensity normalized by the dye concentration), and the relative photoacoustic signal intensity (relative value of photoacoustic signal intensity normalized by the dye concentration) of the resulting NP-3 containing IgG (hereafter abbreviated as NP-3-IgG) are shown in Table 3.

Preparation of Composite Particle in which IgG is Bound to Surface of Particle Including Polyfunctional Dye

In order to bind IgG to the surface of NP-3 obtained as described above, initially, glycine (final concentration 1 mM) was added to the NP-3 solution, and agitation was performed at room temperature for 1 hour. Thereafter, glycine was removed by using a PD-10 desalting column (produced by GE Healthcare Bioscience).

Subsequently, bis(sulfosuccinimidyl) suberate (BS3, produced by Thermo Fisher Scientific K.K.) was added to the particle suspension liquid. Here, the reaction was effected while the reaction molar ratio (the number of moles of fed BS3/the number of moles of particle including iron oxide particle) was specified to be 100,000. After the resulting particle suspension liquid was agitated at room temperature for 30 minutes, the PD-10 desalting column (produced by GE Healthcare Bioscience) was used and, thereby, NP-3, in which a surface amino group was NHS-esterified, and an unreacted BS3 were separated. Then, a composite particle was prepared by binding IgG in the same manner as that in Example 2 (Preparation of composite particle (2)) described above.

The amount of binding of dye, the amount of binding of antibody, the relative fluorescence intensity (relative value of fluorescence intensity normalized by the dye concentration), and the relative photoacoustic signal intensity (relative value of photoacoustic signal intensity normalized by the dye concentration) of the resulting NP-3 including IgG on the particle surface (hereafter abbreviated as NP-3(IgG)) are shown in Table 3.

TABLE 3

| | NP-3-IgG | NP-3 (IgG) |
|---|---|---|
| Binding position of IgG | dye | particle surface |
| Amount of binding of dye per particle | 8 | 8 |
| Amount of binding of antibody per particle | 7 | 9 |
| Relative fluorescence intensity | 0.5 | 1.0 |
| Relative photoacoustic signal intensity | 1.7 | 1.0 |

As shown in Table 3, the fluorescence intensity of NP-3-IgG decreased by 50% as compared with that of NP-3(IgG). It was ascertained that in the case where IgG was bound directly to the dye, the fluorescence from the dye was further quenched as compared with that in the case where IgG was bound to the particle surface. The photoacoustic signal intensity of NP-3-IgG was higher than that of NP-3(IgG), and the signal intensity 1.7 times that of NP-3(IgG) was obtained. It was shown that direct boning of IgG to the dye was advantageous for reduction in fluorescence quantum yield of the dye and increase in heat-confining effect and, as a result, it is believed to be advantageous for increase in photoacoustic signal intensity.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-290328 filed Dec. 27, 2010, which is hereby incorporated by reference herein in its entirety.

What is claimed is:

1. A composite particle comprising:
a particle containing a first dye having a molar absorption coefficient of 10 M⁻¹cm⁻¹ or more at least one wavelength selected within the range of 600 nm to 1,300 nm;
a second dye which is bound to the surface of the particle and which has a molar absorption coefficient of 10 M⁻¹cm⁻¹ or more at least one wavelength selected within the range of 600 nm to 1,300 nm; and
an antibody,
wherein the antibody is bound to the second dye.

2. The composite particle according to claim 1, wherein the first dye is different from the second dye.

3. The composite particle according to claim 1, wherein the first dye is an inorganic material and the second dye is an organic dye.

4. The composite particle according to claim 1, wherein the first dye is an iron oxide particle.

5. The composite particle according to claim 1, wherein the second dye is an organic dye having at least two reactive functional groups.

6. A contrast agent for photoacoustic imaging, comprising:
the composite particle according to claim 1; and
a dispersion medium.

7. A photoacoustic imaging method comprising the steps of:
irradiating a specimen, which has been administrated with the contrast agent for photoacoustic imaging according to claim 6, with the light in a wavelength region of 600 nm to 1,300 nm, and
detecting an acoustic wave emitted from the contrast agent present in the specimen.

8. A composite particle comprising a particle containing an inorganic material, an organic dye bound to the particle, and an antibody, wherein the antibody is bound to the organic dye.
